(19) United States
(12) Patent Application Publication
(10) Pub. No.: US 2004/0138598 A1
(43) Pub. Date: Jul. 15, 2004

(54) Cold pack finger splint

(75) Inventors: Marnie A. Kortuem, Tracy, MN (US); Ivan E. Brown, Spirit Lake, IA (US); Teryle L. Kounkel, Spirit Lake, IA (US); Rebecca A. Mills, Spirit Lake, IA (US); Shanna Marks, Spirit Lake, IA (US)

Correspondence Address: MCKEE, VOORHEES & SEASE, P.L.C., 801 GRAND AVENUE, SUITE 3200, DES MOINES, IA, 50309-2721, US

(73) Assignee: Brown Medical Industries, Spirit Lake, IA

(21) Appl. No.: 10/341,557

(22) Filed: Jan. 13, 2003

(51) Int. Cl. A61F 5/00

(52) U.S. Cl. 602/22

(57) ABSTRACT

A finger splint, ice pack gel combination, using a finger splint with an associated ice gel pack so that both support and cold therapy can simultaneously be applied to an injured finger.

### FIELD OF THE INVENTION

The present invention relates generally to finger splints and more specifically to a finger splint that can simultaneously provide support for an injured finger and cold therapy to aid in relief from pain and swelling.

### BACKGROUND OF THE INVENTION

Finger splints to protect injured fingers have, of course, been known for years. Typically, such finger splints can be foam splints of the wrap around type, fold-over type, prong splints, or gutter splints. They can be made from metal or plastic.

Constructional features of these conventional finger splints are well known. However, they have application problems. In particular, they are designed for finger protection and support in an immobile position, without necessarily providing comfort or assistance in reducing swelling and pain. As well, such conventional finger splints are often cumbersome, uncomfortable to wear and thus discourage patient compliance.

There is therefore a need for a finger splint that not only protects and immobilizes but also assists in reduction of pain and swelling.

It is accordingly an object of the present invention to provide an improved finger splint which provides protection and finger immobilization along with cold therapy for reduction of swelling and pain. The result is quicker healing to allow the injured person to more immediately return to their normal activities of work or play.

Another object of the present invention is to provide an ice pack sheathed splint that will overcome the shortcomings of the prior art finger splints.

A further object of the present invention is to provide an ice pack in conjunction with a splint for providing instant relief from painful finger injuries.

Yet another object is to provide a finger splint ice pack configuration that will serve a dual purpose for injured digits by providing support and protection, as well as a cooling component to minimize pain and swelling.

Another object is to provide a finger splint that will be an affordable way for coaches/clinicians to stabilize an injury until proper medical treatment can be sought.

Another object is to provide a finger splint that any individual without medical skill will be able to apply and adjust with ease.

A still further object is to provide a finger splint that provides a custom fit for fingers of different shapes and sizes due to its flexible, cushioning gel sheath that enshrouds the splint.

Another object is to provide a finger splint that will stand up to uses in many different environments without breaking.

Other objects and advantages of the present invention will become apparent from the written description and it is intended that these objects and advantages are all within the scope of the present invention.

To accomplish the above and related objects, this invention may be embodied in the form illustrated in the accompanying drawings; however, the drawings are illustrative only, and changes may be made in the specific constructions illustrated which are preferred embodiments only.

### SUMMARY OF THE INVENTION

In its broadest sense, the invention comprises a finger splint/ice gel pack combination. The combination employs a finger splint of any of the conventional types having an associated ice gel pack so that both support and protection, and ice therapy for pain and swelling, can simultaneously be applied to an injured finger. The invention also relates to specific constructional features embodying this concept in a manner that is both economical and will aid in patient compliance. Such constructional details are described in the preferred embodiment set forth below.

### BRIEF DESCRIPTION OF THE DRAWINGS

FIG. 1 is a perspective view of a preferred prong finger splint embodiment of the invention sheathed in a gel wrap or ice pack.

FIG. 2 is a side view of the device of FIG. 1 along line 2-2.

FIG. 3 is an end view of one of the prongs of the device of FIG. 1, along lines 3-3.

FIG. 4 is an in-use view of the prong finger splint of FIG. 1.

FIG. 5 is a bottom view of a wrap-around splint, with the ice pack.

FIG. 6 is an in-use view of a fold-over splint, with the ice pack.

FIG. 7 is an in-use view of a gutter splint, with the ice pack.

### DETAILED DESCRIPTION OF A PREFERRED EMBODIMENT

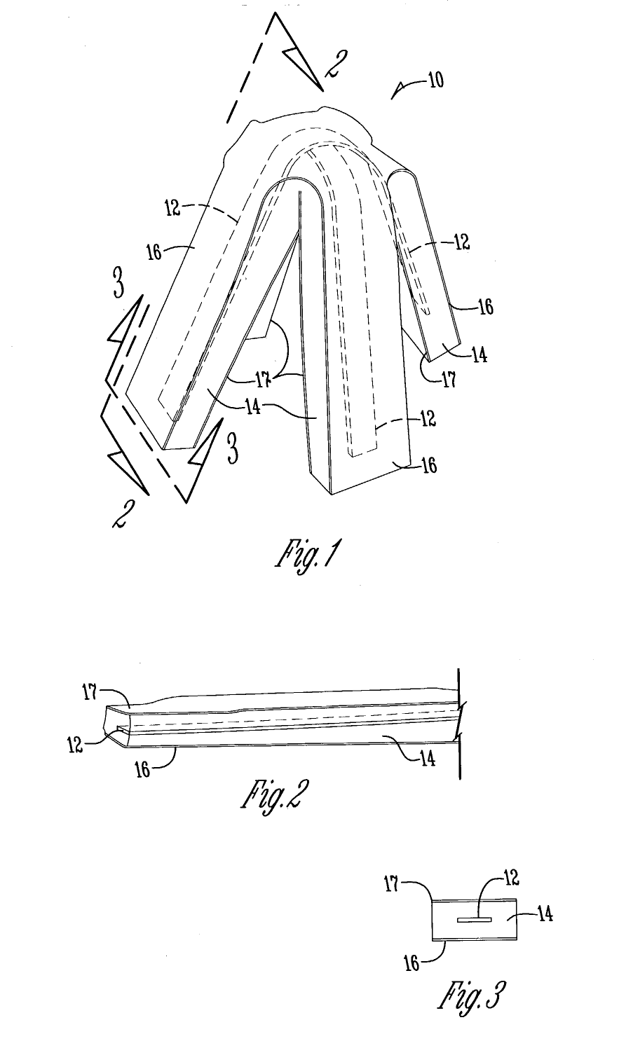

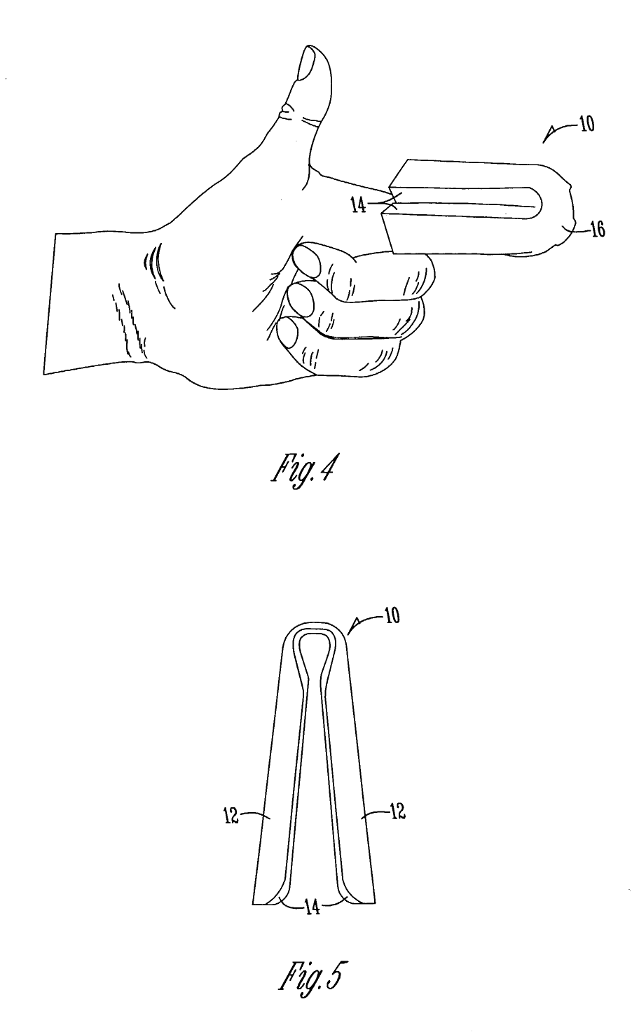

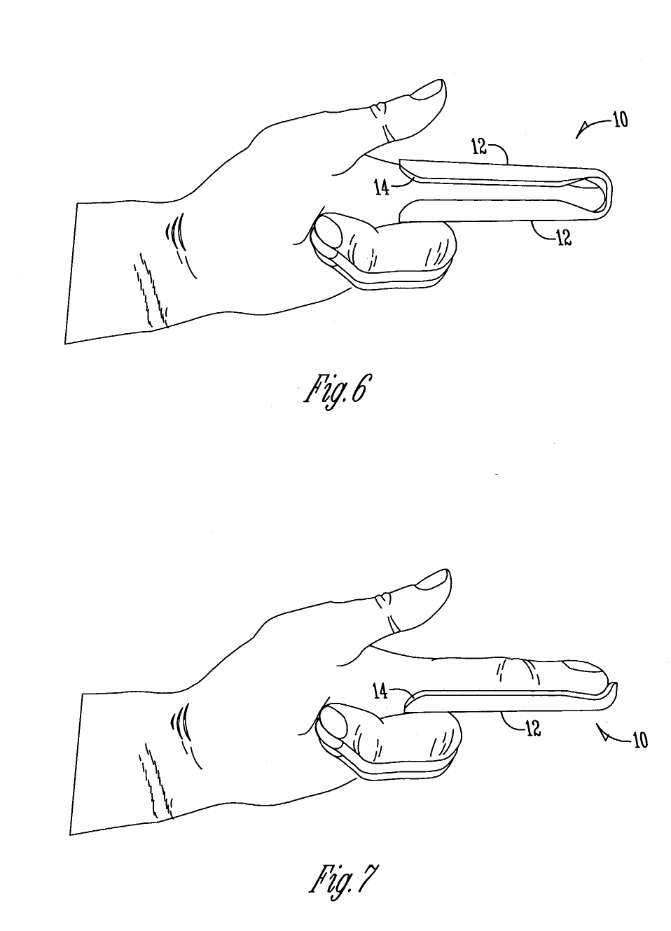

Turning now to the drawings in which similar reference characters denote similar elements through the several views, illustrated in FIGS. 1-7 is the combination of various finger splint shells such as a fold-over splint (FIG. 6), a gutter splint (FIG. 7), prong splint (FIG. 1-4) and a wrap-around splint (FIG. 5), with the preferred configuration shown in the detail of FIGS. 1-4, which configuration will be described with particularity.

The finger splint ice pack/gel combination, referred to generally as 10 can use a prong splint 12 as depicted in FIG. 1 at 12, or a wrap-around splint, a gutter splint or a fold-over splint. Each of the splint shells 12 are available in the art and each are preferred by some users. They all serve the purpose of protection and immobilization. Shell splint 12 can be made of a plastic polymeric material which can be polyethylene or polypropylene, or it can be made of a metal such as malleable aluminum. Malleable aluminum is preferred.

The finger splint shell 12 is encased, enshrouded or embedded in a sheath of surrounding gel material 14 (see FIG. 1). The sheath of gel material 14 is an inherently adhering plastisol material. It is covered with an outer thin layer 16 of cotton, nylon or even gauze and an inner layer 17. Gel pack 14 is a thermal gel material, adherent, soft and compliant, with a spongy feel. The gel material 16 is commercially available. One suitable gel material 14 is sold by Three G Corporation of 110 West 3^{rd} North Street, Morristown, Tenn. 37814. Such gel material need not be described with chemical particularity except to say that such materials are Plastisols and present a soft cushioning feel, even when subjected to freezing temperatures. They maintain cold temperatures when exposed to ambient 20° C. temperatures for at least 30 minutes, and in some cases, 60 minutes. Other therapeutic gel materials may be employed, for example the gel material referenced in commonly owned U.S. Pat. No. 6,267,742 relating to a night splint suspension system that can be used in combination with Sealed Ice™. Material safety data sheets are available from Three G Corporation for the Plastisol compound suitable for use in the present invention.

One of the advantages of the thermal gel material 14 is that it may be used unchilled to provide soft, cushioned extraordinary comfort; or, it can be chilled to provide sustained cold therapy for pain and swelling. A reversible gel refers to one that can be used either ambient, warm or cold.

The splint 10 is manufactured in the following way. An outer gauze pad 16 is laid on a tray. Melted therapy gel is poured over it and the splint 12 in flat conditioned is layered over the gel covered gauze pad. Next, another layer of gel material 14 is poured over splint 12, and another gauze layer 17 is placed on top. When the splint 10 is cooled it is then moved to a cutting die and trimmed into shape and bent.

While the above description has been provided in particular reference to the preferred embodiment of a sheathed thermal gel surrounding a finger splint shell, it can, for example, be used in other ways. That is to say, the gel strip can be a lining placed on the inside of the finger splint shell if preferred. In this embodiment, the gel strip lining would adhere to the shell main component on an interior side, and being soft and compliant, it will mold to the various and sundry shaped digits inserted in the finger splint.

With respect to the above description then, it is to be realized that the optimum dimensional relationships for the parts of the invention, include variations in size, materials, shape, form, function and manner of operation, assembly and use, and all are deemed within the scope of the invention. All equivalent relationships to those illustrated in the drawings and described in the specification are intended to be encompassed by the present invention.

In use, the splint 10 is removed from its package, usually cold, and then placed over and around the injured finger and taped into position. The splint 10 provides support and protection and the gel 14 supplies cold therapy and comfort and cushioning

As used herein, the terms “cold pack”, or “ice gel pack” or “ice pack”, refer to the temperature of the gel pack and not its composition. Put another way, the material is not referred to as “ice” because of water content but because of its temperature, i.e. ice cold. That is not to say, however, that in some cases, a composition having a water constituent could not be employed. Proper combinations of gel ingredients may make this possible.

What is claimed is:

1. A finger splint ice gel pack combination comprising:
a finger splint having an associated ice-gel pack so that both support and cold therapy can simultaneously be applied to a finger in need of such treatment.

2. The finger splint of claim 1 wherein the finger splint is a shell splint.

3. The finger splint of claim 1 wherein the finger splint is a fold over shell splint.

4. The finger splint of claim 1 wherein the finger splint is a gutter splint.

5. The finger splint of claim 1 wherein the finger splint is an aluminum finger splint.

6. The finger splint of claim 1 wherein the finger splint is a polymeric plastic finger splint.

7. The finger splint of claim 1 wherein the ice-gel pack enshrouds the finger splint.

8. A finger splint gel pack combination, comprising:
a finger splint sheathed in a reversible gel ice pack.

9. The finger splint of claim 8 wherein the finger splint is enclosed in a thin liner material.

10. The finger splint of claim 8 wherein the finger splint is a finger splint shell selected from the group consisting of wrap-around splint shells, fold-over splint shells, gutter splint shells and pronged splint shells.

11. The finger splint of claim 10 wherein the finger splint shell is fold over finger splint shell.

12. A finger splint, comprising:
a finger splint having an interior surface and an exterior surface;
said interior surface being associated with an ice pack liner to provide cold therapy while said finger splint provides finger support.

13. A method of treating an injured digit, comprising:
placing said digit in a finger splint having an associated ice-gel pack so that both support and cold therapy can simultaneously be applied to a finger in need of such treatment.
